(19) United States
(12) Patent Application Publication
(10) Pub. No.: US 2017/0081484 A1
(43) Pub. Date: Mar. 23, 2017

(54) A PROCESS OF MANUFACTURING DIPPED LATEX PRODUCTS WITHOUT USING ANIMAL DERIVATIVES AND PRODUCTS THEREOF

(71) Applicant: TTK Protective Devices Limited, Chennai (IN)

(72) Inventors: Srinivasan Jagannathan, Chennai (IN); N. Balasubramanian, Chennai (IN)

(21) Appl. No.: 15/126,158

(22) PCT Filed: Jun. 15, 2015
(86) PCT No.: PCT/IN2015/000242

§ 371 Date: Sep. 14, 2016

(30) Foreign Application Priority Data
Jun. 13, 2014 (IN) 2908/CHE/2014

(51) Int. Cl. C08J 5/02; C08K 3/34; C08K 3/06; A61F 6/04; A61L 31/02; B29C 41/14; B29C 35/02; B29C 41/00; C08J 3/26; A61L 31/04

(52) U.S. Cl. CPC C08J 5/02; C08J 3/26; C08K 3/346; C08K 3/06; A61L 31/049; A61L 31/028; B29C 41/14; B29C 35/0227; B29C 41/003; A61F 6/04; C08J 2307/02; B29K 2105/0064

(57) ABSTRACT

The present disclosure provides for a process for manufacturing of dipped latex products without using any animal derived ingredients and dipped latex products produced by said process. The disclosed process successfully over comes drawbacks of drop in product yield (due to coagulum formation) and burst pressures caused by absence-of casein in latex formulation. Therefore, the dipped latex products so produced through the process have final composition similar to that of any other dipped latex products with casein except for absence of casein and having 0.8 phr of Sulphur as against 0.6 phr of Sulphur in routine manufacturing. The products also exhibit physical properties similar to that of any other dipped latex products with casein, such as burst pressure and burst volume.

### FIELD OF THE INVENTION

The present invention relates to the field of manufacturing latex products. More particularly the present invention relates to a process of manufacturing dipped latex products without using animal derivatives.

### BACKGROUND OF THE INVENTION

Dipped latex products such as gloves, condoms, balloons and finger cots are made by well-established manufacturing process where the curing agent typically Sulphur, accelerators, activators, surfactants are added to one or more lattices, namely: Natural Rubber Lattices including Hevea brasiliensis and Guayule to prepare a liquid compound used in dipping process.

In dipping process, all the compounding ingredients and curing agents typically Sulphur, accelerators, activators, surfactants are added to lattices in the manufacturing stage called compounding. As per the state of the art, all the compounding ingredients must be mixed homogenously in the lattices to produce the dipped articles with good physical properties.

The latex procured from rubber plantations need to have some selected additives such as curing agents typically Sulphur, accelerators, activators, surfactants, protein etc. added so that the desired physical properties can be obtained in the finished product. One such additive is casein—a protein derived from cow's milk (animal protein)—which is added as a thickener or binding agent at the stage of preparation of rubber compound. The casein mainly acts as (i) a binding agent for several ingredients in the latex compound and (ii) as a thickener that controls viscosity of the latex compound during the coating process.

In case of manufacturing inflatable rubber products such as balloons, condoms, gloves etc. from natural lattices, absence of casein from the latex formulation leads to a reduction in burst pressure of about 0.3 kPa. Usually the condoms with casein in the routine formulation used in the existing manufacturing process achieve burst pressures of about:
1.6 kPa for 65 micron thick condoms, and
1.5 kPa for 55 micron thick condoms.

The minimum requirement of ISO 4074:2014 is burst pressure of 1 kPa in the condoms. When casein is removed, the burst pressures range between 1.2 kPa to 1.3 kPa. At this range of initial pressures the risk of encountering failures increases making it difficult to comply with the requirements of ISO 4074:2014. Removing casein also makes the latex less stable increasing the amount of coagulum formed. The problems encountered due to removal of casein in the formulation while manufacturing dipped latex products such as, but not limited to, condoms are:
Drop in product yield by about 20% due to coagulum formation, and
Impossible to make reliable thinner condoms (55 microns) as the burst pressures drop too close to the permissible limit to confidently meet ISO 4074:2002 requirements.

However as a matter of personal principle or lifestyle choice a segment of users prefer that the products they use are free from animal derived materials including casein.

Therefore, there is a specific need for latex products devoid of any type of animal derivatives which could be used by everybody without any hesitation.

### SUMMARY OF THE INVENTION

The present disclosure overcomes aforementioned problems encountered due to removal of casein in the formulation while manufacturing dipped latex products.

In one aspect of the present invention, a process of manufacturing dipped latex products without using animal derivatives is provided. The steps of the process include preparing Sulphur paste by mixing Sulphur, demineralized water, Bentonite clay and predetermined quantity of one or more dispersants while maintaining Sulphur level at 0.8 phr; compounding natural rubber latex with at least one of one or more surfactants with one or more diluents, the Sulphur paste and accelerator paste; dipping one or more formers into the compounded natural rubber latex to form a plurality of coatings of the compounded natural rubber latex on the dipped one or more formers; and curing the plurality of coatings on the formers to form the dipped latex products. During the compounding natural rubber latex pre-vulcanization is carried out at temperature ranging from 40° C. to 60° C. and for a duration ranging from 24 hours to 48 hours. Further, quantity of the one or more surfactants added during compounding is adjusted as per the predetermined quantity of the one or more dispersants added to the Sulphur paste.

In further aspect of the present invention, additionally soy protein is mixed to the natural rubber latex during compounding. The plant based protein is added to compensate the absence of casein during the compounding of the natural rubber latex.

Other features of the embodiments will be apparent from the detailed description that follows.

### DETAILED DESCRIPTION OF THE INVENTION

Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular. The materials, methods, and examples are illustrative only and not intended to be limiting. It must be noted that, as used in the specification and the appended claims, the singular forms “a,” “an” and “the” include plural referents unless the context clearly dictates otherwise.

The terms “compounded latex” and “latex formulation” have been synonymously used throughout the description. Further, the term “green” in the context of the present invention is used interchangeably with “vegan”, “sustainable”, “environment—friendly”, “non-exploitative”, “ethically produced”. The term “green process” in the context of the present invention implies the process of manufacturing of dipped latex products without using any animal derived ingredients disclosed in the present specification, while or “green latex products/condoms” implies a latex product/condom manufactured by the present process.

The present invention provides a process for manufacturing of dipped latex products without using any animal derived ingredients and dipped latex products produced by said process. The process is used in manufacturing including, but not limited to, condom, glove, balloon, catheter, finger cot, surgical tubing, baby bottle nipple, and dental dam that are free from casein protein and/or any other animal derived ingredients.

The present process (green process) does not involve use of the casein or any other animal derived product. It successfully over comes drawbacks of drop in product yield (due to coagulum formation) and burst pressures caused by absence of casein in latex formulation. Therefore, the dipped latex products so produced through the process have final composition similar to that of any other dipped latex products with casein except for absence of casein and having 0.8 phr of Sulphur as against 0.6 phr of Sulphur in routine manufacturing. The products also exhibit physical properties similar to that of any other dipped latex products with casein, such as burst pressure and elasticity.

For manufacturing green dipped latex products natural rubber latex drawn from Heavea brasiliensis is used which is obtained from a source certified by Forest Stewardship Council (FSC). This is to ensure that source/supplier providing the natural rubber latex must have the latex bearing trees cultivated in an environmental friendly method, no child labour being used and that the labour be used in a non-exploitative way. The entire process must be sustainable both from point of view of environment as well as human/labour factors.

The present invention overcomes the aforementioned drawbacks broadly by adjusting levels of surfactants and curing agents in compounded latex by at least one of the following:
a) Increasing the amount of Sulphur in the latex formulation,
b) Altering the stages of addition of the surfactants (surfactant paste), and
c) Altering surfactant level in the Sulphur paste preparation stage.

The process of manufacturing the dipped latex products without using animal derivatives includes following steps:

(A) Preparing Sulphur paste (Vulcanizing agent) by mixing Sulphur, demineralized water, Bentonite clay and predetermined quantity of dispersant(s). This stage plays a critical role in the process and subsequent formulation of latex.

An extra quantity of Sulphur is added to the Sulphur paste so as to maintain its level at 0.8 phr (which is higher as compared to 0.6 phr of Sulphur level maintained in the state of the art).

The predetermined quantity dispersant(s) added to the Sulphur paste is also higher as compared to the quantity of surfactants added as per existing state of the art. It is to be noted that the addition of extra dispersant(s) in sulphur paste preparation stage rather than at the compound preparation stage is significant deviation from the existing state of the art. The dispersant(s) are added in less quantity in later stages so as to balance overall quantity of dispersant(s) in the compounded latex. The negative effects of increased sulphur are off-set by dispersion techniques. The dispersant(s) are selected from a group consisting of Vultamol, Emulgin, Cetodet and Potassium hydroxide.

The Sulphur paste includes following ingredients in the given proportion:

45 to 55% by weight Sulphur,

44 to 50% by weight demineralized water,

0.005 to 0.1% by weight Bentonite clay

4 to 10% by weight one or more dispersants.

(B) Compounding natural rubber latex with at least one of surfactant(s) with diluent(s), the Sulphur paste and accelerator paste. The quantity of the surfactant(s) added during compounding is adjusted as per the predetermined quantity of the dispersant(s) added to the Sulphur paste. This is done so that the final composition of the latex product is comparable to similar “non-green” latex products except for using FSC latex and being free from animal derived materials. The pH of the compounded latex is adjusted to a range of about 9 to 11.5.

The surfactant(s) are selected from a group consisting of Potassium oleate solution, Potassium hydroxide solution, Emulgin B2, Cetodet 500 and Vulcastab LW Solution. The diluent(s) used with the surfactant(s) are selected from a group consisting of potassium hydroxide solution, demineralized water and ammoniated DM water.

The accelerator paste includes accelerator agent(s) and demineralized water. The accelerator agent(s) are selected from a group consisting Vultamol, Zinc dibutyl dithiocarbamate, Irganox and ZnO.

During the step of compounding, pre-vulcanisation is carried out at a lower temperature (as compared to the existing state of the art) ranging from 40° C. to 60° C. and for a longer duration (as compared to the existing state of the art) ranging from 24 hours to 48 hours. However, the extra Sulphur probably did not have sufficient temperature at this stage to result in damages such as over-curing of the liquid latex compound and rendering it unfit to deliver required physical properties in the product finishing stage.

These changes do not affect the final composition of the condoms except for removal of casein and having 0.8 phr of Sulphur as against 0.6 phr of Sulphur in routine manufacturing. More specifically, where thinner condoms are to be made the Sulphur phr is increased in the latex compound.

A high level of isolation and de-contamination of materials used is maintained thought out the step. The specified latex received from a certified supplier is held isolated to prevent any contaminations/mixing with any other latex or latex compounds. The required surfactants, Sulphur pastes, accelerator pastes for the latex compound preparation are made separately to be free from animal derived material and are held separately until required for compound preparation.

The compounding includes following ingredients in the given proportion:

90 to 95% by weight natural rubber latex;

5 to 8% by weight one or more surfactants with one or more diluents;

0.6 to 0.8% by weight the Sulphur paste; and

0.6 to 0.8% by weight accelerator paste.

(C) Subsequent to the compounding of the natural rubber latex dipping is performed, where formers/moulds are dipped into the compounded natural rubber latex to form a plurality of coatings of the compounded natural rubber latex on the dipped formers.

The compounded natural rubber latex is taken to a designated dipping plant specially prepared with thorough cleaning measures to prevent contaminations and is filled in dipping vessels in the plant and dipping is carried out. In one of the embodiments of manufacturing condoms by the present process, the number of dipping vessels are 2. However, in alternate embodiments the number of vessels may vary depending on required′ thickness of the condoms. Similarly, the number of dippings carried out in dipping vessels and subsequent formation of the coatings of the compounded natural rubber latex also varies depending on the required thickness of the product to be formed, such as but not limited to condoms. The temperature of the compounded latex is maintained within 25±5° C. during the step of dipping.

(D) Finally Curing the plurality of coatings on the formers to form the dipped latex products. The plurality of coatings of the compounded natural rubber latex on the dipped former(s) are dried in a hot vulcanizing oven maintained within a range of 60° C. to 120° C.

Once the curing is completed, leaching is performed on the dried former(s) and subsequently product from the formers are stripped, wherein the process of leaching and stripping are similar to those employed in the manufacture of other natural latex products such as, but not limited to, condoms.

In the embodiment of manufacturing condoms by the present process, the condoms so produced after curing step are kept isolated for further processing of testing and foil-packing to be carried out in designated testing and foil packing machines at a later stage to ensure highest reliability and quality.

As discussed previously that the present invention requires extra precaution for avoiding any sort of contamination and following are guidelines are followed throughout the process—starting from receipt of raw latex, chemicals compound preparation, compounded latex preparation, dipping, post-treatment, electronic testing and foil packing as well as final packing
i. Cleaning of the vessels used to hold the received latex,
ii. Cleaning of the vessels used to prepare latex compounding chemicals,
iii. Cleaning and de-contamination of the chemicals milling equipment such as attritors, stirrers etc. In this context de-contamination is carried out by washing insides of pipe lines, valves, milling apparatus parts that cannot be reached by hand/visually by using alkali solutions and soap solutions. For example alkali solutions are pumped through the milling apparatus to completely remove any previous process residues. Similarly stirrers are run with alkali/soap solutions to remove all traces of any previous process residues.
iv. Isolation of raw materials, chemicals in process, compounded latex, dipped latex products/condoms, tested latex products/condoms, foil-packed and final packed latex products/condoms are carried out by labeling the containers and physically holding them separately from similar process materials of other normal latex and latex products/condoms produced by the green process.
v. Cleaning and de-contamination of the dipping plant inclusive of the dippers, leach tanks, glass formers, bead forming brushes, other washing brushes, post-treatment stage equipment etc. are similarly carried out using soap solutions, alkali solutions before starting the dipping process for “green” latex products/condoms. In case of glass formers acids are used to clean.
vi. Similarly the electronic testing and foil packing equipment and their accessories are subjected to thorough cleaning and isolation prior to starting “green” process.

In a further embodiment the process of manufacturing the dipped latex products without using animal derivatives, plant based protein such as soy protein is added to compensate the absence of casein during the compounding of the natural rubber latex.

During the step of compounding following ingredients were added in given proportion:

90 to 95% by weight natural rubber latex

0.05 to 0.2% by weight soy protein

5 to 8% by weight one or more surfactants with one or more diluents

0.6 to 0.75% by weight the Sulphur paste

0.6 to 0.8% by weight accelerator paste

In one of the embodiments of the present invention, three types of condoms are made as described below:
1) Type MT02G: Flat width 49 mm, thickness 65 microns, parallel, smooth finish
2) Type NF01AG: Contoured shape, thickness 65 microns, smooth finish
3) Type MT03AG: Flat width 52 mm, parallel smooth finish, thickness 55 microns.

These three types of condoms were subjected to the following tests to verify if their physical properties were stable and continued to comply to industry standards as well as international standards (for example ISO 4074):
1) Accelerated ageing at 70° C. for duration upto 28 days;
2) Accelerated ageing at 50° C. for duration upto 6 months; and
3) Real-time ageing (room temperature) for duration upto 9 months.

Three batches of condoms were drawn from each type and were foil-packed dosed with suitable lubricant and kept in ovens held at 50° C. and at 70° C. At pre-set time-points determined by ISO 4074 standards for condoms, samples were drawn from the ovens and physical properties tests (air-burst tests) were carried out. It is known in the chemical industry that raised temperature simulates ageing. For example when a product is stored at 50° C. for 6 months the effect on the product would be same as for holding at 30° C. for 3 years. The temperature and duration are recommended by relevant sections of ISO 4074 standards. The standards recommend a minimum burst pressure of 1 kPa and burst volume of 18 litres at all conditions.

### EXAMPLES

The present invention is explained further in the following specific examples which are only by way of illustration and are not to be construed as limiting the scope of the invention.

### Example 1

### Type MT02G: Flat Width 49 mm, Thickness 65 Microns, Parallel, Smooth Finish

This condom is a smooth textured natural rubber latex condom made using Forest Stewardship Council certified latex. The compounding process onwards is free from any animal derived ingredients. The condom had a flat width of 49 mm, a length of 180 mm and thickness of 65 microns.

The results of the accelerated stability tests and comparison with real-time stability tests for three batches of this type of condoms are tabulated below:

### Burst Pressure

TABLE 1
Burst pressure values - accelerated ageing at 70° C.
(Pressure in kPa)

| | | Pressure at 70° C. Accelerated temperature | | | | |
|---|---|---|---|---|---|---|
| Product type | | condition and duration in kPa | | | | |
| MT02G | | Initial | 7 days/70° C. | 14 days/70° C. | 21 days/70° C. | 28 days/70° C. |
| 49 mm, 65 | batch 1 | 2.04 | 1.96 | 1.93 | 1.93 | 1.73 |
| microns, | batch 2 | 2.10 | 2.00 | 1.99 | 1.98 | 1.85 |
| parallel, | batch 3 | 2.11 | 2.09 | 2.00 | 2.04 | 1.85 |
| smooth | | | | | | |

TABLE 2
Burst pressure values - accelerated ageing at 50° C.
(Pressure in kPa)

| | | Pressure at 50° C. Accelerated temperature | | | | |
|---|---|---|---|---|---|---|
| Product type | | condition and duration in kPa | | | | |
| MT02G | | Initial | 1 m/50° C. | 2 m/50° C. | 3 m/50° C. | 6 m/50° C. |
| 49 mm, 65 | batch 1 | 2.04 | 2.06 | 1.95 | 1.96 | 2.02 |
| microns, | batch 2 | 2.10 | 2.14 | 2.08 | 2.13 | 2.02 |
| parallel, | batch 3 | 2.11 | 2.15 | 2.09 | 2.07 | 2.04 |
| smooth | | | | | | |

TABLE 3
Burst pressure values - Real-time ageing at room temperature
(Pressure in kPa)

| | | Pressure at room temperature (real time) | | | | | | | |
|---|---|---|---|---|---|---|---|---|---|
| Product type | | condition and duration in kPa | | | | | | | |
| MT02G | | Initial | 1 m/RT | 2 m/RT | 3 m/RT | 4 m/RT | 5 m/RT | 6 m/RT | 9 m/RT |
| 49 mm, 65 | batch 1 | 2.04 | 2.11 | 2.02 | 2.05 | 2.04 | 2.02 | 2.07 | 1.96 |
| microns, | batch 2 | 2.10 | 2.13 | 2.09 | 2.13 | 2.05 | 2.06 | 2.05 | 1.98 |
| parallel, | batch 3 | 2.11 | 2.14 | 2.08 | 2.14 | 2.00 | 2.07 | 2.16 | 2.01 |
| smooth | | | | | | | | | |

By examining the trend of pressure over different time points at different conditions it is noted that the pressure remains steady, does not indicate any abnormal degradation/decrease and is well above the minimum value of 1 kPa specified by international standards.

At any condition the condoms continue to have stable physical properties. Overall we can confidently claim that these condoms would have the same shelf-life as condoms made in the routine process.

### Burst Volume

TABLE 4
Burst Volume values - accelerated ageing at 70° C.
(Volume in Litres)

| | | Volume at 70° C. Accelerated temperature | | | | |
|---|---|---|---|---|---|---|
| | | condition and duration in Litres | | | | |
| MT02G | | Initial | 7 d/70° C. | 14 d/70° C. | 21 d/70° C. | 28 d/70° C. |
| 49 mm, 65 | batch 1 | 33.33 | 29.6 | 27 | 25.7 | 30.4 |
| microns, | batch 2 | 33.96 | 30.3 | 30.2 | 28.1 | 30.2 |
| parallel, | batch 3 | 34.81 | 28.3 | 33.2 | 26.5 | 29.4 |
| smooth | | | | | | |

TABLE 5
Burst Volume values - accelerated ageing at 50° C.
(Volume in Litres)

| | | Volume at 50° C. Accelerated temperature | | | | |
|---|---|---|---|---|---|---|
| | | condition and duration (Litres) | | | | |
| MT02G | | Initial | 1 m/50° C. | 2 m/50° C. | 3 m/50° C. | 6 m/50° C. |
| 49 mm, 65 | batch 1 | 33.33 | 31.8 | 29.6 | 28.5 | 26.2 |
| microns, | batch 2 | 33.96 | 29.8 | 29.8 | 26.7 | 28.7 |
| parallel, | batch 3 | 34.81 | 30.5 | 29.3 | 28.4 | 28.9 |
| smooth | | | | | | |

TABLE 6
Burst Volume values - Real-time ageing at room temperature
(Volume in Litres)

| | | Volume at room temperature (real time) | | | | | | | |
|---|---|---|---|---|---|---|---|---|---|
| | | condition and duration (Litres) | | | | | | | |
| MT02G | | Initial | 1 m/RT | 2 m/RT | 3 m/RT | 4 m/RT | 5 m/RT | 6 m/RT | 9 m/RT |
| 49 mm, 65 | batch 1 | 33.33 | 31.7 | 33.8 | 30.1 | 33.7 | 34 | 28.7 | 32.5 |
| microns, | batch 2 | 33.96 | 33.2 | 34.2 | 31.2 | 34.1 | 31.3 | 31.5 | 33.1 |
| parallel, | batch 3 | 34.81 | 33.3 | 33.8 | 30.1 | 33.6 | 33.4 | 30.4 | 33.7 |
| smooth | | | | | | | | | |

By examining the trend of volume over different time points at different conditions it is noted that the volume remains steady, does not indicate any abnormal degradation/decrease and is well above the minimum value of 18 litres specified by international standards.

At any condition the condoms continue to have stable physical properties. Overall we can confidently claim that these condoms would have the same shelf-life as condoms made in the routine process.

### Example 2

### Type NF01AG: Contoured Shape, Thickness 65 Microns, Smooth Finish

This condom is a smooth textured natural rubber latex condom made using Forest Stewardship Council certified latex. The compounding process onwards is free from any animal derived ingredients. The condom has a contoured shape for a comfortable fit, a length of 180 mm and thickness of 65 microns.

The results of the accelerated stability tests and comparison with real-time stability tests for three batches of this type of condoms are tabulated below:

### Burst Pressure

TABLE 7
Burst pressure values - accelerated ageing at 70° C.
(Pressure in kPa)

| | | Pressure at 70° C. Accelerated temperature | | | | |
|---|---|---|---|---|---|---|
| | | condition and duration in kPa | | | | |
| NF01AG | | Initial | 7 d/70° C. | 14 d/70° C. | 21 d/70° C. | 28 d/70° C. |
| Contour, | batch 1 | 1.93 | 1.85 | 1.77 | 1.75 | 1.77 |
| 65 mic, | batch 2 | 1.90 | 1.84 | 1.81 | 1.78 | 1.77 |
| smooth | batch 3 | 1.93 | 1.81 | 1.85 | 1.78 | 1.78 |

TABLE 8
Burst pressure values - accelerated ageing at 50° C.
(Pressure in kPa)

| | | Pressure at 50° C. Accelerated temperature | | | | |
|---|---|---|---|---|---|---|
| | | condition and duration in kPa | | | | |
| NF01AG | | Initial | 1 m/50° C. | 2 m/50° C. | 3 m/50° C. | 6 m/50° C. |
| Contour, | batch 1 | 1.93 | 1.99 | 1.87 | 1.91 | 1.94 |
| 65 mic, | batch 2 | 1.90 | 1.97 | 1.88 | 1.92 | 1.96 |
| smooth | batch 3 | 1.93 | 1.98 | 1.84 | 1.96 | 1.96 |

TABLE 9
Burst pressure values - Real-time ageing at room temperature
(Pressure in kPa)

| | | Pressure at room temperature (real time) | | | | | | | |
|---|---|---|---|---|---|---|---|---|---|
| | | condition and duration in kPa | | | | | | | |
| NF01AG | | Initial | 1 m/RT | 2 m/RT | 3 m/RT | 4 m/RT | 5 m/RT | 6 m/RT | 9 m/RT |
| Contour, | batch 1 | 1.93 | 1.96 | 1.96 | 1.88 | 1.86 | 1.92 | 1.99 | 1.95 |
| 65 mic, | batch 2 | 1.90 | 1.93 | 1.94 | 1.98 | 1.90 | 1.89 | 2.01 | 1.94 |
| smooth | batch 3 | 1.93 | 1.87 | 1.96 | 2.01 | 1.85 | 1.90 | 1.98 | 1.92 |

By examining the trend of pressure over different time points at different conditions it is noted that the pressure remains steady, does not indicate any abnormal degradation/decrease and is well above the minimum value of 1 kPa specified by international standards.

At any condition the condoms continue to have stable physical properties. Overall we can confidently claim that these condoms would have the same shelf-life as condoms made in the routine process.

### Burst Volume

TABLE 10
Burst Volume values - accelerated ageing at 70° C.
(Volume in Litres)

| | | Volume at 70° C. Accelerated temperature | | | | |
|---|---|---|---|---|---|---|
| | | condition and duration in Litres | | | | |
| NF01AG | | Initial | 7 d/70° C. | 14 d/70° C. | 21 d/70° C. | 28 d/70° C. |
| Contour, | batch 1 | 41.69 | 34.5 | 32.7 | 33.3 | 33.5 |
| 65 mic, | batch 2 | 40.64 | 33.6 | 32.3 | 32.8 | 33.8 |
| smooth | batch 3 | 40.09 | 35.3 | 32.8 | 32.9 | 33.7 |

TABLE 11
Burst Volume values - accelerated ageing at 50° C.
(Volume in Litres)

| | | Volume at 50° C. Accelerated temperature | | | | |
|---|---|---|---|---|---|---|
| | | condition and duration in Litres | | | | |
| NF01AG | | Initial | 1 m/50° C. | 2 m/50° C. | 3 m/50° C. | 6 m/50° C. |
| Contour, | batch 1 | 41.69 | 33.3 | 36.7 | 33.4 | 29.9 |
| 65 mic, | batch 2 | 40.64 | 32.1 | 37.1 | 34.2 | 29.1 |
| smooth | batch 3 | 40.09 | 31.8 | 36.5 | 29.1 | 29.3 |

TABLE 12
Burst Volume values - Real-time ageing at room temperature
(Volume in Litres)

| | | Volume at room temperature (real time) | | | | | | | |
|---|---|---|---|---|---|---|---|---|---|
| | | condition and duration (Litres) | | | | | | | |
| NF01AG | | Initial | 1 m/RT | 2 m/RT | 3 m/RT | 4 m/RT | 5 m/RT | 6 m/RT | 9 m/RT |
| Contour, | batch 1 | 41.69 | 40.1 | 34.1 | 37.3 | 41.1 | 42.4 | 36.2 | 39.2 |
| 65 mic, | batch 2 | 40.64 | 38.8 | 32.6 | 33.3 | 41.5 | 41 | 34.6 | 40.2 |
| smooth | batch 3 | 40.09 | 36.9 | 34 | 32.9 | 41.4 | 40.8 | 35.2 | 39.6 |

By examining the trend of volume over different time points at different conditions it is noted that the volume remains steady, does not indicate any abnormal degradation/decrease and is well above the minimum value of 18 litres specified by international standards.

At any condition the condoms continue to have stable physical properties. Overall we can confidently claim that these condoms would have the same shelf-life as condoms made in the routine process.

### Example 3

### Type MT03AG: Flat Width 52 mm, Parallel Smooth Finish, Thickness 55 Microns

This condom is a smooth textured natural rubber latex condom made using Forest Stewardship Council certified latex. The compounding process onwards is free from any animal derived ingredients. The condom has a flat width of 52 mm, a length of 180 mm and thickness of 55 microns.

The results of the accelerated stability tests and comparison with real-time stability tests for three batches of this type of condoms are tabulated below:

### Burst Pressure

TABLE 13
Burst pressure values - accelerated ageing at 70° C.
(Pressure in kPa)

| MT03AG | | Initial | 7 d/70° C. | 14 d/70° C. | 21 d/70° C. | 28 d/70° C. |
|---|---|---|---|---|---|---|
| 52 mm, | batch 1 | 1.88 | 1.88 | 1.86 | 1.93 | 1.75 |
| 55 mic, | batch 2 | 1.87 | 1.91 | 1.85 | 1.90 | 1.76 |
| parallel | batch 3 | 1.98 | 2.06 | 2.02 | 1.96 | 1.81 |
| smooth. | | | | | | |

TABLE 14
Burst pressure values - accelerated ageing at 50° C.
(Pressure in kPa)

| MT03AG | | Initial | 1 m/50° C. | 2 m/50° C. | 3 m/50° C. | 6 m/50° C. |
|---|---|---|---|---|---|---|
| 52 mm, | batch 1 | 1.88 | 1.87 | 1.73 | 1.87 | 1.91 |
| 55 mic, | batch 2 | 1.87 | 1.82 | 1.70 | 1.90 | 1.86 |
| parallel | batch 3 | 1.98 | 2.14 | 1.69 | 2.00 | 1.88 |
| smooth. | | | | | | |

TABLE 15
Burst pressure values - Real-time ageing at room temperature
(Pressure in kPa)

| MT03AG | | Initial | 1 m/RT | 2 m/RT | 3 m/RT | 4 m/RT | 5 m/RT | 6 m/RT | 9 m/RT |
|---|---|---|---|---|---|---|---|---|---|
| 52 mm, | batch 1 | 1.88 | 1.92 | 1.90 | 1.85 | 2.00 | 2.00 | 1.88 | 1.94 |
| 55 mic, | batch 2 | 1.87 | 1.91 | 1.94 | 1.94 | 1.92 | 1.95 | 1.87 | 1.95 |
| parallel | batch 3 | 1.98 | 2.09 | 2.07 | 2.04 | 2.00 | 2.02 | 2.06 | 1.93 |
| smooth. | | | | | | | | | |

By examining the trend of pressure over different time points at different conditions it is noted that the pressure remains steady, does not indicate any abnormal degradation/decrease and is well above the minimum value of 1 kPa specified by international standards.

At any condition the condoms continue to have stable physical properties. Overall we can confidently claim that these condoms would have the same shelf-life as condoms made in the routine process.

### Burst Volume

TABLE 16
Burst Volume values - accelerated ageing at 70° C.
(Volume in Litres)

| MT03AG | | Initial | 7 d/70° C. | 14 d/70° C. | 21 d/70° C. | 28 d/70° C. |
|---|---|---|---|---|---|---|
| 52 mm, | batch 1 | 32.87 | 26.3 | 26.4 | 24.9 | 29.8 |
| 55 mic, | batch 2 | 34.95 | 26.6 | 27.3 | 24.8 | 29.6 |
| parallel | batch 3 | 34.72 | 25.8 | 26.4 | 24.9 | 30.5 |
| smooth. | | | | | | |

TABLE 17
Burst Volume values - accelerated ageing at 50° C.
(Volume in Litres)

| MT03AG | | Initial | 1 m/50° C. | 2 m/50° C. | 3 m/50° C. | 6 m/50° C. |
|---|---|---|---|---|---|---|
| 52 mm, | batch 1 | 32.87 | 30.7 | 30.7 | 26 | 23.7 |
| 55 mic, | batch 2 | 34.95 | 33.1 | 30.7 | 26.1 | 29.8 |
| parallel | batch 3 | 34.72 | 24.4 | 29.7 | 26.2 | 27.2 |
| smooth. | | | | | | |

TABLE 18
Burst Volume values - Real-time ageing at room temperature
(Volume in Litres)

| MT03AG | | Initial | 1 m/RT | 2 m/RT | 3 m/RT | 4 m/RT | 5 m/RT | 6 m/RT | 9 m/RT |
|---|---|---|---|---|---|---|---|---|---|
| 52 mm, | batch 1 | 32.87 | 29.9 | 30.6 | 31.4 | 33.8 | 33.2 | 28.8 | 33.5 |
| 55 mic, | batch 2 | 34.95 | 28.6 | 30.3 | 32 | 33.2 | 33.7 | 28.5 | 33.3 |
| parallel | batch 3 | 34.72 | 28 | 30 | 31.2 | 34.2 | 33.8 | 28.7 | 32.1 |
| smooth. | | | | | | | | | |

By examining the trend of volume over different time points at different conditions it is noted that the volume remains steady, does not indicate any abnormal degradation/decrease and is well above the minimum value of 18 litres specified by international standards.

At any condition the condoms continue to have stable physical properties. Overall we can confidently claim that these condoms would have the same shelf-life as condoms made in the routine process.

The above examples and the results obtained substantiate the reliability, effectiveness and quality of “Green” condoms manufactured by the present process.

The details of the comparison of “green condoms” devoid of any animal derived ingredient as against “normal” condoms are listed in Table 19 with significant differences highlighted in bold.

TABLE 19
Comparison of Compositions of the “normal” and “green” condoms

| S. | Chemicals used in formulation and | Chemicals used in formulation of |
|---|---|---|
| No | composition of “normal” product | proposed “Green” product |
| 1 | Basic material | |
| | Natural Rubber Latex drawn from | Natural Rubber Latex drawn from |
| | Heavea brasiliensi | Heavea brasiliensis with FSC |
| | | certification |
| 2 | Surfactants and diluents | |
| | Demineralised Water | Demineralised Water (DM water) |
| | Ammoniated DM Water/DM | Ammoniated DM Water/DM |
| | Water | Water |
| | Potassium Oleate Solution | Potassium Oleate Solution |
| | Emulgin B2/Cetodet 500/ | Emulgin B2/Cetodet 500/ |
| | Vulcastab LW Solution | Vulcastab LW Solution |
| | Potassium Hydroxide Solution | Potassium Hydroxide Solution |
| 3 | Vulcanising agents/Sulphur paste | |
| | Sulphur at 0.6 phr | Sulphur at 0.8 phr |
| | DM water | DM water |
| | Vultamol | Vultamol |
| | Bentonite clay | Bentonite clay |
| | Sulphur | Sulphur |
| | Ammonium casienate solution | Not used. |
| | | Instead extra Vultamol/Emulgin/ |
| | | Cetodet/Potassium hydroxide |
| | | used as required. Same quantity |
| | | removed from other stages. |
| 4 | Accelerator agents/Composite paste | |
| | Vultamol | Vultamol |
| | Ammonium caseinate solution | Not used |
| | ZDBC | ZDBC |
| | Irganox | Irganox |
| | ZnO | ZnO |
| | DM water | DM water |
| 5 | Compounding process | |
| | For natural latex compound | The natural latex compound |
| | preparation. Pre-vulcanisation is | preparation includes maintaining |
| | performed at temperature of 60° C. | and using the formulation of |
| | | chemicals as described above |
| | | and carry out pre-vulcanisation |
| | | at 40° C. to 60° C. |
| | The batch size of latex compound | The batch size is limited to |
| | could be upto 2000 kg. This | approximately one to two days' |
| | quantity of compounded latex is | dipping requirement-less than |
| | meant to be used for several days' | 500 kg. This is to limit the level of |
| | dipping process. | pre-vulcanisation since the sulphur |
| | | quantity is higher in this |
| | | formulation |
| 7 | Dipping and Post treatment process. | |
| | Standard compound Dipping | Dipping Process performed while |
| | process | maintaining and using the |
| | | Natural latex compound |
| | | prepared as described above |
| 8 | Electronic Testing (ET) | |
| | Standard prescribed for natural | Standard prescribed for natural |
| | latex Condoms ET process | latex Condoms ET process. |
| 9 | Foil packing/lubricant dosing | |
| | Standard Condom foiling process. | Standard Condom foiling process |
| | Depending upon customer | is followed while for lubrication |
| | specification may use Silicone | using pure silicone fluid without |
| | lubricant with flavours/ | any flavour/masking additives. |
| | masking agents as additives. | |
| 10 | Final packing | |
| | As per Customer specification and | As per Customer specification and |
| | market standard | market standard |

While the invention has been described in detail with reference to preferred embodiments thereof, it will be apparent to one skilled in the art that various changes can be made, and equivalents employed, without departing from the scope of the invention.

### GLOSSARY OF TERMS AND DEFINITIONS

Physical properties of the finished products are determined by how much of the sulphur has formed cross links within and between the polyisoprene chains or the lattices. For Lattices this process is termed as “vulcanization”.

The term “pre-vulcanization” is used to refer to the vulcanization taking place at the stage of preparing liquid compound using the lattices. The term “curing” is generally used in place of “vulcanization” in the document.

A “dispersant” or a dispersing agent or a plasticizer or a superplasticizer is either a non-surface active polymer or a surface-active substance added to a suspension, usually a colloid, to improve the separation of particles and to prevent settling or clumping. The dispersants consist normally of one or more surfactants.

We claim:

1. A process of manufacturing dipped latex products without using animal derivatives, comprising:
preparing Sulphur paste by mixing Sulphur, demineralized water, Bentonite clay and predetermined quantity of one or more dispersants while maintaining Sulphur level at 0.8 phr;
compounding natural rubber latex with at least one of one or more surfactants with one or more diluents, the Sulphur paste and accelerator paste, wherein
the natural rubber latex is drawn from Heavea brasiliensis,
pre-vulcanization is carried out at temperature ranging from 40° C. to 60° C. and for a duration ranging from 24 hours to 48 hours, and
quantity of the one or more surfactants added during compounding being adjusted as per the predetermined quantity of the one or more dispersants added to the Sulphur paste;
dipping one or more formers into the compounded natural rubber latex to form a plurality of coatings of the compounded natural rubber latex on the dipped one or more formers; and
curing the plurality of coatings on the formers to form the dipped latex products.

2. The process as claimed in claim 1, further comprising adding soy protein during the compounding of the natural rubber latex.

3. The process as claimed in claim 1, wherein the one or more dispersants are selected from a group consisting of Vultamol, Emulgin, Cetodet and Potassium hydroxide.

4. The process as claimed in claim 1, wherein the one or more surfactants are selected from a group consisting of Potassium oleate solution, Potassium hydroxide solution, Emulgin, Cetodet and Vulcastab LW Solution.

5. The process as claimed in claim 1, wherein the one or more diluents are selected from a group consisting of potassium hydroxide solution, demineralized water and ammoniated DM water.

6. The process as claimed in claim 1, wherein the accelerator paste comprises one or more accelerator agents and demineralized water.

7. The process as claimed in claim 6, wherein the one or more accelerator agents are selected from a group consisting Vultamol, Zinc dibutyl dithiocarbamate, Irganox and ZnO.

8. The process as claimed in claim 1, wherein pH of the compounded latex is adjusted to a range of about 9 to 11.5.

9. The process as claimed in claim 1, wherein the temperature of the compounded latex is maintained within 25±5° C. during the dipping.

10. The process as claimed in claim 1, wherein the plurality of coatings of the compounded natural rubber latex on the dipped one or more formers are dried in a hot oven maintained within a range of 60° C. to 120° C.

11. The process as claimed in claim 1, wherein the entire process is carried out in isolation from similar lattices to prevent cross-contamination.

12. A dipped latex product devoid of any animal derivative obtained through the process of claim 1.

13. The dipped latex product as claimed in claim 12, wherein the dipped latex product is a condom, glove, balloon, catheter, finger cot, surgical tubing, baby bottle nipple or dental dam.
